(19) United States
(12) Patent Application Publication
(10) Pub. No.: US 2021/0236376 A1
(43) Pub. Date: Aug. 5, 2021

(54) SMART WALKER SYSTEM AND METHOD OF USE THEREOF

(71) Applicant: Lauren SIEGEL, Rock Hill, SC (US)

(72) Inventor: Lauren SIEGEL, Rock Hill, SC (US)

(21) Appl. No.: 16/879,026

(22) Filed: May 20, 2020

Related U.S. Application Data
(60) Provisional application No. 62/969,182, filed on Feb. 3, 2020.

(51) Int. Cl. A61H 3/04; G16H 40/63; G08B 21/04

(52) U.S. Cl. CPC A61H 3/04; G16H 40/63; G08B 21/0461; A61H 2201/0188; A61H 2201/5012; A61H 2201/5058; A61H 2201/0184; G08B 21/0446

(57) ABSTRACT

A smart walker system includes a walker attachment and a user proximity sensor. The walker attachment is configured to be attached to the medical assistance walker. The user proximity sensor is in communication with the walker attachment on the medical assistance walker for determining a proximity of the user proximity sensor relative to the walker attachment on the medical assistance walker. The user proximity sensor is configured to be worn by a user. The user proximity sensor is configured to detect when the user moves away from the medical assistance walker a set distance. Wherein, when the user wearing the user proximity sensor moves the set distance away from the walker attachment on the medical assistance walker, the smart walker system is configured to alert the user to use the medical assistance walker.

### CROSS-REFERENCE TO RELATED APPLICATIONS

This application claims benefit of priority application U.S. Provisional Ser. No. 62/969,182 filed on Feb. 3, 2020, entitled “Smart Walker” which is incorporated herein by reference in its entirety.

### FIELD OF THE DISCLOSURE

The present disclosure is related to tools for disabled people, like a walker. More specifically, the present disclosure relates to a smart walker system and method of use thereof.

### BACKGROUND

Generally speaking a walker or walking frame is a tool for disabled people, who need additional support to maintain balance or stability while walking, most commonly due to age-related physical restrictions. Walkers started appearing in the early 1950s. The first US patent was awarded in 1953 to William Cribbes Robb, of Stretford, UK, for a device called “walking aid”, which had been filed with the British patent office in August 1949. Two variants with wheels were both awarded US patents in May 1957, and the first non-wheeled design that was called a “walker” was patented in 1965 by Elmer F. Ries of Cincinnati, Ohio. The first walker to resemble modern walkers was patented in 1970 by Alfred A. Smith of Van Nuys, Calif.

The basic design consists of a lightweight frame that is about waist high, approximately 12 inches (30 cm) deep and slightly wider than the user. Walkers are also available in other sizes such as pediatric for children or bariatric for obese persons. Modern walkers are height adjustable and should be set at a height that is comfortable for the user, but will allow the user to maintain a slight bend in their arms. This bend is needed to allow for proper blood circulation through the arms as the walker is used. The front two legs of the walker may or may not have wheels attached, depending on the strength and abilities of the person using it. It is also common to see caster wheels or glides on the back legs of a walker with wheels on the front; additionally felt products adapted to glide with the legs such as adhesive feet, along with tennis balls with holes cut into them to place them onto the legs are also utilized on surfaces such as hardwood, epoxy and linoleum flooring common to institutions.

A different approach to the walker is the rollator, also called wheeled walker, invented by the Swede Aina Wifalk in 1978, herself a polio sufferer. Although originally a brand name, “rollator” has become a genericized trademark for wheeled walkers in many countries, and is also the most common type of walker in several European countries. The rollator consists of a frame with three or four large wheels, handlebars and a built-in seat, which allows the user to stop and rest when needed. Rollators are also often equipped with a shopping basket. Rollators are typically more sophisticated than conventional walkers with wheels. They are adjustable in height and are light-weight, yet sturdier than conventional walkers. The handlebars are equipped with hand brakes that can be lifted or pushed downward to instantly stop the rollator. The brakes can also be used in maneuvering the rollator; by braking one side while turning the rollator towards that side a much tighter turning radius can be achieved.

In use, the person walks with the frame surrounding their front and sides and their hands provide additional support by holding on to the top of the sides of the frame. Traditionally, a walker is picked up and placed a short distance ahead of the user. The user then walks to it and repeats the process. With the use of wheels and glides, the user may push the walker ahead as opposed to picking it up. This makes for easier use of the walker, as it does not require the user to use their arms to lift the walker. This is beneficial for those with little arm strength. A walker is often used by those who are recuperating from leg or back injuries. It is also commonly used by persons having problems with walking or with mild balance problems.

One problem that the instant disclosure addresses is that users of walkers may forget to use their walker and walk off on their own, or may purposely not use the walker as prescribed. Obviously, this may create a dangerous situation where the disabled person is susceptible to falling and can therefore lead to an injury or worse. Therefore, a need exists for a walker or walker system that may notify the user and/or a third party, like a nurse, care taker or relative that the user has left their walker behind.

The instant disclosure may be designed to address at least certain aspects of the problems or needs discussed above by providing a smart walker system and method of use thereof.

### SUMMARY

The present disclosure may solve the aforementioned limitations of the currently available walkers and users thereof, by providing a smart walker system. The disclosed smart walker system may generally include a walker attachment and a user proximity sensor. The walker attachment is configured to be attached to the medical assistance walker. The user proximity sensor is in communication with the walker attachment on the medical assistance walker for determining a proximity of the user proximity sensor relative to the walker attachment on the medical assistance walker. The user proximity sensor is configured to be worn by a user. The user proximity sensor is configured to detect when the user moves away from the medical assistance walker a set distance. Wherein, when the user wearing the user proximity sensor moves the set distance away from the walker attachment on the medical assistance walker, the smart walker system is configured to alert the user to use the medical assistance walker.

One feature of the disclosed smart walker system may be that it can be configured to promote the use of the medical assistance walker by the user.

Another feature of the disclosed smart walker system may be that it can be configured to promote the use of the medical assistance walker to prevent accidental falls.

In select embodiments of the disclosed smart walker system, the user proximity sensor may be configured to be worn around a neck of the user. In select embodiments, the user proximity sensor may include a wearable device housing a proximity sensor. The proximity sensor may be in communication with the walker attachment on the medical assistance walker. A flexible elongated member may be included and connected at a first side of the wearable device on a first end and connected to a second side of the wearable device on a second end. In select embodiments, the wearable device may include an oval shaped housing. The oval shaped housing of the wearable device may include a first light configured for indicating a status of the smart walker system.

In select embodiments of the disclosed smart walker system, the walker attachment may be configured to attach to one of a plurality of support members of the medical assistance walker. In select possibly preferred embodiments of the walker attachment, it can be configured to attach to a top front cross member of one of the plurality of support members of the medical assistance walker. The walker attachment may be configured to be retrofitted to an existing medical assistance walker or it may be included or built into a new medical assistance walker. In select embodiments, the walker attachment may include an attachment means configured for attaching the walker attachment to the top front cross member. The attachment means may include a hinged back member with hinges configured to wrap around the top front cross member of the medical assistance walker. A key lock may also be included and may be configured to lock the hinged back member around the top front cross member of the medical assistance walker. In select embodiments, the walker attachment may include a rectangular housing. The rectangular housing may include a second light, a third light, a proximity distance adjuster, and/or a system display. The second light in the rectangular housing may be configured for indicating a device connection status. The third light in the rectangular housing may be configured for indicating a device disconnected status. The proximity distance adjuster may be configured for adjusting the set distance. The system display may be configured to display information of the smart walker system.

In select embodiments of the disclosed smart walker system, a charger may be included. The charger may be for charging the walker attachment, and/or the user proximity sensor. In select embodiments, the charger may include a power cord, a fourth light, a fifth light, a first charging port, a second charging port, a sixth light, and/or a seventh light. The power cord may have a standard plug configured for attaching to an electrical outlet. The fourth light may be for indicating charging. The fifth light may be for indicating power to the charger. The first charging port may be configured for connecting the walker attachment to the charger for charging. The second charging port may be configured for connecting the user proximity sensor to the charger for charging. The sixth light may be configured for indicating the status of the walker attachment. The seventh light may be configured for indicating the status of the user proximity sensor.

Another feature of the disclosed smart walker system may be that it can be configured to remind the user to use the medical assistance walker, to alert nursing stations, to alert smart home devices, to notify a mobile care taker of a subscription mobile app, to notify life alert, and/or to notify a home alarm product. In select embodiments, the walker attachment may include a Wi-Fi communication device configured to communicate over an internet with the nursing stations, to alert the smart home devices, to notify the mobile care taker of the subscription mobile app, to notify the life alert, and/or to notify the home alarm product.

In select embodiments of the disclosed smart walker system a subscription mobile app may be included for alerting a mobile care taker. The subscription mobile app may have a user interface. The user interface may include an about us tab, a login tab, a products tab, a help tab, and/or a visual indication of an active walker or an inactive walker.

In another aspect, the instant disclosure embraces the smart walker system in any of the various embodiments and/or combination of embodiments shown and/or described herein.

In another aspect, the instant disclosure embraces a method of alerting a user to use a medical assistance walker. The disclosed method of alerting a user to use a medical assistance walker generally includes utilizing the disclosed smart walker system in any of the various embodiments and/or combination of embodiments shown and/or described herein. As such, in select embodiments, the method of alerting a user to use a medical assistance walker may include: attaching a walker attachment to the medical assistance walker; wearing a user proximity sensor by the user; determining a proximity of the user with the user proximity sensor relative to the medical assistance walker with the walker attachment; detecting when the user moves away from the medical assistance walker a set distance; and alerting the user to use the medical assistance walker.

The foregoing illustrative summary, as well as other exemplary objectives and/or advantages of the disclosure, and the manner in which the same are accomplished, are further explained within the following detailed description and its accompanying drawings.

### BRIEF DESCRIPTION OF THE DRAWINGS

The present disclosure will be better understood by reading the Detailed Description with reference to the accompanying drawings, which are not necessarily drawn to scale, and in which like reference numerals denote similar structure and refer to like elements throughout, and in which:

FIG. 1 shows an environmental perspective view of one embodiment of the smart walker system according to the instant disclosure;

FIG. 2 shows an environmental perspective view of another embodiment of the smart walker system according to the instant disclosure communicating with a nurses station;

FIG. 3 shows an environmental perspective view of another embodiment of the smart walker system according to the instant disclosure communication with various components through the internet;

FIG. 4 shows a schematic representation of the charger for the disclosed smart walker system according to select embodiments of the instant disclosure;

FIG. 5 shows a schematic representation of a walker attachment for the disclosed smart walker system according to select embodiments of the instant disclosure;

FIG. 6 shows a schematic representation of a user proximity sensor for the disclosed smart walker system according to select embodiments of the instant disclosure;

FIG. 7 shows a schematic representation of a screen shot of a mobile application for the disclosed smart walker system according to select embodiments of the instant disclosure; and

FIG. 8 shows a flow chart of a method of alerting a user to use their medical assistance walker according to select embodiments of the instant disclosure.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

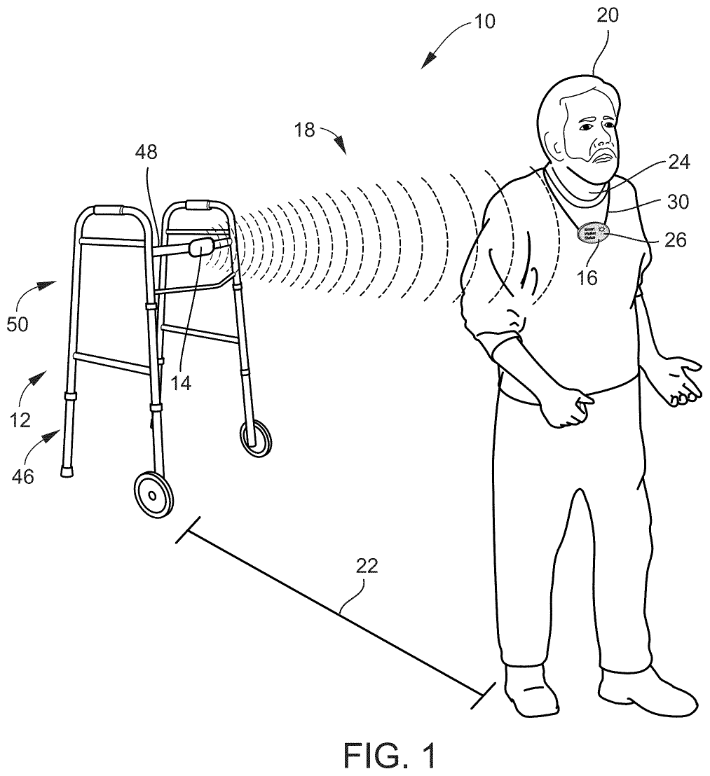

Referring now to FIGS. 1-8, in describing the exemplary embodiments of the present disclosure, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples and are merely examples among other possible examples.

Referring now to FIGS. 1-7, the present disclosure solves the aforementioned limitations of the currently available devices and methods of medical assistance walkers and use thereof by providing smart walker system 10. Smart walker system 10 may be for alerting and/or reminding a user to use their medical assistance walker 12 or to alert other people or care givers that the user is not using their medical assistance walker 12. Smart walker system 10 may be an add-on system for retrofitting an existing medical assistance walker 12 or it may be included or built into a new medical assistance walker 12. Smart walker system 10 may generally include walker attachment 14 and user proximity sensor 16. Walker attachment 14 may be configured to be attached to medical assistance walker 12. User proximity sensor may be in communication with walker attachment 14 on medical assistance walker 12 for determining proximity 18 of user proximity sensor 16 relative to walker attachment 14 on medical assistance walker 12. User proximity sensor 16 may be configured to be worn by user 20. User proximity sensor 16 may be configured to detect when user 20 moves away from medical assistance walker 12 set distance 22. Wherein, when user 20 wearing user proximity sensor 16 moves set distance 22 away from walker attachment 14 on medical assistance walker 12, smart walker system 10 may be configured to alert user 20 to use medical assistance walker 12.

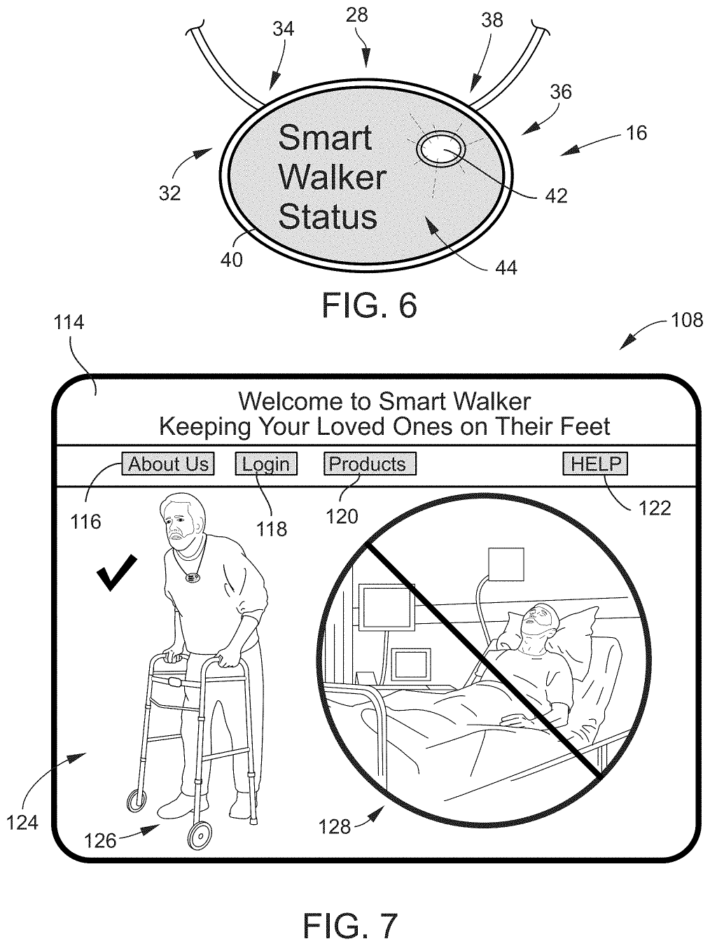

User proximity sensor 16 may be included with smart walker system 10. User proximity sensor 16 may be for detecting proximity 18 or how close user 20 is to their medical assistance walker 12 equipped with walker attachment 14. User proximity sensor 16 may include any devices, mechanisms, members or means for determining proximity 18 or how close user 20 is to their medical assistance walker 12 equipped with walker attachment 14. As sown in the Figures, in select embodiments of smart walker system 10, user proximity sensor 16 may be configured to be worn around neck 24 of user 20, like as a necklace or lanyard. See FIGS. 1-3 and 6-7. However, the disclosure is not so limited and user proximity sensor 16 may be worn anywhere on user 20, like on their wrist as a bracelet, on their clothing as a pin or lanyard, on their ankle as an ankle bracelet, the like, or any other locations on or even embedded in user 20. In select embodiments, user proximity sensor 16 may include wearable device 26. Wearable device 26 may be a housing configured for housing proximity sensor 28. Proximity sensor 28 may be in communication with walker attachment 14 on medical assistance walker 12. Proximity sensor 28 may be any device or means for determining proximity 18 of user proximity device 16 to walker attachment 14, including but not limited to: capacitive, apacitive displacement sensor, doppler effect (sensor based on doppler effect), inductive, magnetic, including magnetic proximity fuse, optical, photoelectric, photocell (reflective), laser rangefinder, passive (such as charge-coupled devices), passive thermal infrared, radar, reflection of ionizing radiation, sonar (typically active or passive), ultrasonic sensor, fiber optics sensor, hall effect sensor, the like, etc. Flexible elongated member 30 may be included for supporting user proximity sensor 16 around neck 24 of user 20. Flexible elongated member may be connected at first side 32 of wearable device 26 on first end 34 and connected to second side 36 of wearable device 26 on second end 38, as best shown in FIG. 6. Flexible elongated member 30 may be any necklace type device including any ropes, strings, chains, elastic members, the like, etc. In select embodiments, wearable device 26 may include oval shaped housing 40, as shown in the Figures. However, the disclosure is not so limited and any design, shape or size of wearable device 26 may be included. In select embodiments, oval shaped housing 40 of wearable device 26 may include first light 42. First light 42 may be configured for indicating status 44 of smart walker system 10, as shown in the figures.

Walker attachment 14 may be included with smart walker system 10. Walker attachment 14 may be for providing the location relative to user proximity sensor 16, i.e. proximity 18. Walker attachment 14 may be configured to attach to medical assistance walker 12 by any means or at any location. In select embodiments, walker attachment 14 may be configured to attach to one of a plurality of support members 46 of medical assistance walker 12. In select possibly preferred embodiments, walker attachment 14 can be configured to attach to top front cross member 48 of one of the plurality of support members 46 of medical assistance walker 12, as shown in the Figures. Walker attachment 14 may be configured to be retrofitted to existing medical assistance walker 50 (or it may be included or built into a new medical assistance walker 12). In select embodiments, walker attachment 14 may include attachment means 52. See FIG. 5. Attachment means 52 may be configured for attaching walker attachment 14 to top front cross member 48 of any medical assistance walker 12. Attachment means 52 may include any devices, members, mechanisms, means or the like for attaching walker attachment 14 to medical assistance walker 12. In select embodiments, as shown in FIG. 5, attachment means 52 may include hinged back member 54 with hinges 56 configured to wrap around top front cross member 48 of medical assistance walker 12. Key lock 58 may also be included and may be configured to lock hinged back member 54 around top front cross member 48 of medical assistance walker 12. In select embodiments, walker attachment 14 may include rectangular housing 60. However, the disclosure is not so limited and any various size, shape, configuration, or the like of walker attachment 14 may be provided. Rectangular housing 60 may include second light 62, third light 66, proximity distance adjuster 70, and/or system display 72. Second light 62 in rectangular housing 60 may be configured for indicating device connection status 64. Third light 66 in rectangular housing 60 may be configured for indicating device disconnected status 64. Proximity distance adjuster 70 may be configured for adjusting set distance 22. System display 72 may be configured to display information 74 of or relating to smart walker system 10.

Walker attachment 14 and/or user proximity sensor 16 may include a speaker for communicating with user 20 to alert and/or remind them to use medical assistance walker 12. The speaker on walker attachment 14 and/or user proximity sensor 16 may be configured to alert user 20 with a signal or a spoken language. The spoken language broadcast from the speaker may be any desired language and can be configured for the spoken language of user 20, like via subscription mobile app 108. Likewise, subscription mobile app 108 may have a language option that changes the written language to any desired language of mobile care taker 106.

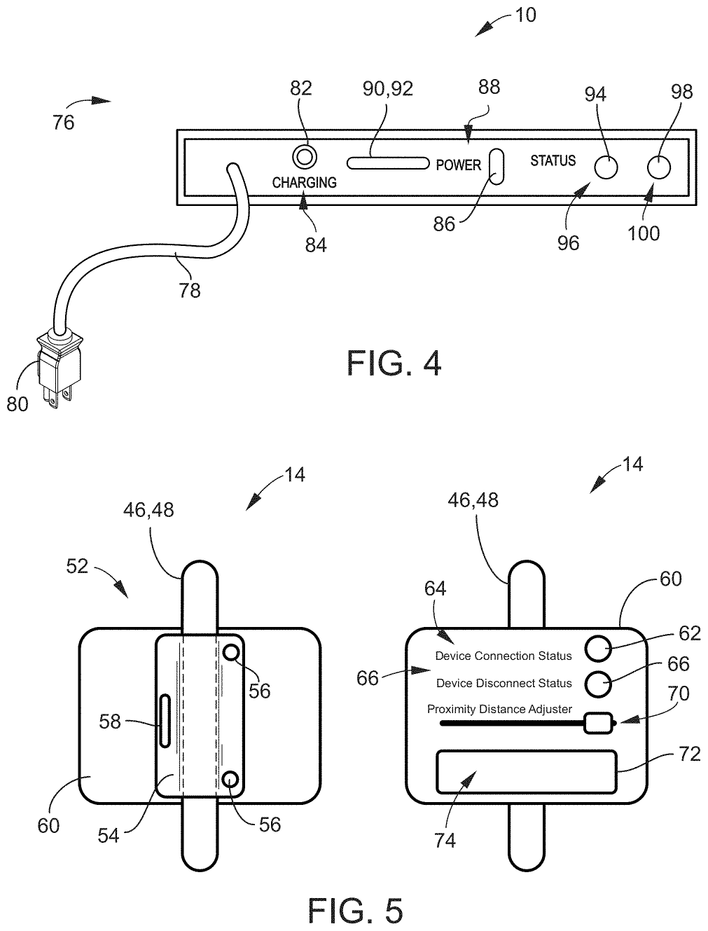

Referring now specifically to FIG. 4, charger 76 may be included with smart walker system 10. Charger 76 may be for charging walker attachment 14 and/or user proximity sensor 16. Charger 76 may include any device or devices, mechanisms, members, means or the like that are configured for charging walker attachment 14, and/or user proximity sensor 16. In select embodiments, as shown in FIG. 4, charger 76 may include power cord 78, fourth light 82, fifth light 86, first charging port 90, second charging port 92, sixth light 94, and/or seventh light 98. Power cord 78 may have standard plug 80 configured for attaching to an electrical outlet for providing power to charger 76. Fourth light 82 may be for indicating charging 84, like for indicating charging of walker attachment 14 and/or user proximity sensor 16. Fifth light 86 may be for indicating power to charger 76, like when power cord 78 is successfully receiving power from standard plug 80 in an electrical outlet. First charging port 90 may be configured for connecting walker attachment 14 to charger 76 for charging. Second charging port 92 may be configured for connecting user proximity sensor 16 to charger 76 for charging. Sixth light 94 may be configured for indicating status 96 of walker attachment 14 (i.e. whether walker attachment 14 is charging or fully charged). Seventh light 98 may be configured for indicating status 100 of user proximity sensor 16 (i.e. whether user proximity sensor 16 is charging or fully charged).

One feature of smart walker system 10 may be that it can be configured to promote the use of medical assistance walker 12 by user 20.

Another feature of smart walker system 10 may be that it can be configured to promote the use of medical assistance walker 12 by user 20 to prevent accidental falls.

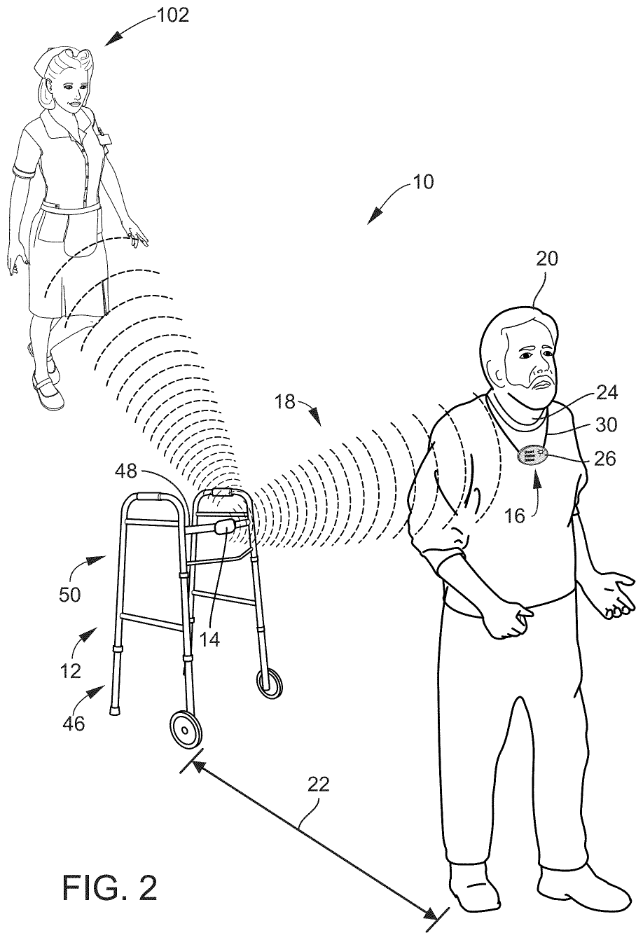

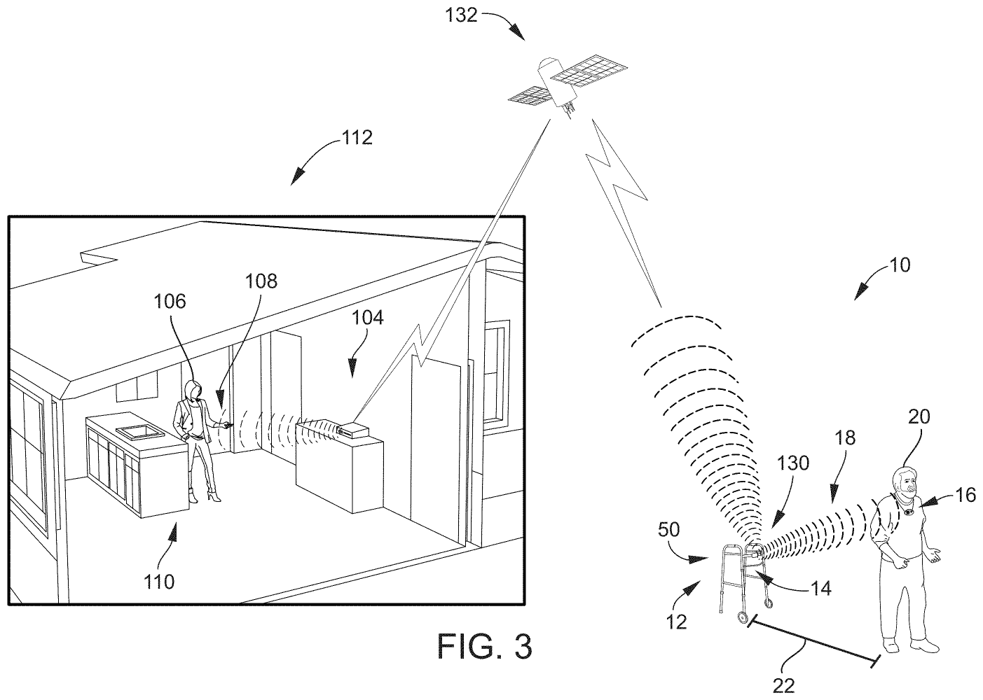

Referring now specifically to FIGS. 2 and 3, another feature of smart walker system 10 may be that it can be configured to remind user 20 to use medical assistance walker 12, to alert nursing stations 102 (see FIG. 2), to alert smart home devices 104 (see FIG. 3), to notify mobile care taker 106 of subscription mobile app 108 (see FIG. 3), to notify life alert 110 (see FIG. 3), and/or to notify home alarm product 112 (see FIG. 3).

Referring now specifically to FIG. 3, in select embodiments of smart walker system 10, Wi-Fi communication device 130 may be included for communicating over internet 132. Wi-Fi communication device 130 may be configured to allow smart walker system 10 to alert any devices or third party care takers of user 20 not using their medical assistance walker 12 after moving farther than set distance 22 from medical assistance walker 12 equipped with walker attachment 14. In select embodiments, as shown in FIG. 3, walker attachment 14 may include Wi-Fi communication device 130 configured to communicate over internet 132, like with nursing stations 102, to alert the smart home devices 104, to notify mobile care taker 106 of the subscription mobile app 108, to notify the life alert 110, and/or to notify the home alarm product 112.

Referring now specifically to FIG. 7, subscription mobile app 108 may be included with smart walker system 10. Subscription mobile app 108 may be any subscription or non subscription app or software for linking mobile care taker 106 with smart walker system 10. As such, subscription mobile app 108 may be included for alerting mobile care taker 106, and/or for linking smart home devices 104, life alert 110, and/or home alarm product 112. Subscription mobile app 108 may have user interface 114. User interface 114 may be configured for interacting with mobile care taker 106 for operating smart walker system 10. User interface 114 may include any setup or configuration for interacting with mobile care taker 106 for operating smart walker system 10. As shown in FIG. 7, in select embodiments, user interface 114 of subscription mobile app 108 may include about us tab 116, login tab 118, products tab 120, help tab 122, and/or visual indication 124. Visual indication 124 may be for indicating the use of smart walker system 10 by user 20. Visual indication 124 may include an indication of an active walker 126 and/or an inactive walker 128, like as shown in FIG. 7.

In another aspect, the instant disclosure embraces the smart walker system in any of the various embodiments and/or combination of embodiments shown and/or described herein.

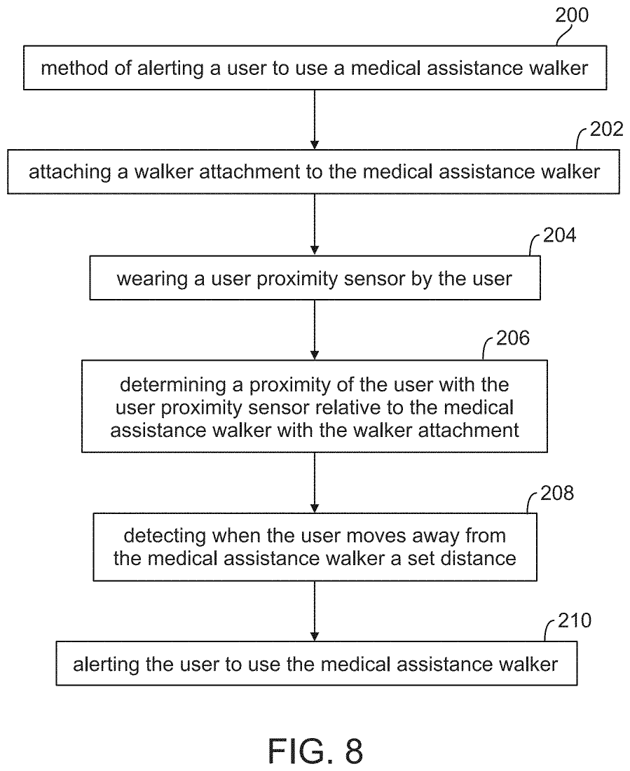

Referring now specifically to FIG. 8, in another aspect, the instant disclosure embraces method 200 of alerting user 20 to use medical assistance walker 12. Method 200 of alerting user 20 to use medical assistance walker 12 may generally include utilizing smart walker system 10 in any of the various embodiments and/or combination of embodiments shown and/or described herein. As such, in select embodiments, method 200 of alerting user 20 to use medical assistance walker 12 may include: step 202 of attaching walker attachment 14 to medical assistance walker 12; step 204 of wearing user proximity sensor 16 by user 20; step 206 of determining proximity 18 of user 20 with user proximity sensor 16 relative to medical assistance walker 12 with walker attachment 14; step 208 of detecting when user 20 moves away from medical assistance walker 12 set distance 22; and step 210 of alerting user 20 to use medical assistance walker 12. However, method 200 is not so limited and may include any other steps for utilizing smart walker system 10 in any of the various embodiments and/or combination of embodiments shown and/or described herein.

In sum, smart walker system 10 may be designed to attach to medical assistance walker 12 and can include a matching user proximity sensor 16 that goes around neck 24 of user 20 of the walker to promote the use of medical assistance walker 12 and prevent accidental falls. If user 20 moves past the proximity setting of a few feet the wearable device 26 on walker would remind user 20 to use their walker until they come back in range of the proximity sensor 28. In addition, walker attachment 14 attached to walker 12 could alert nursing stations 102 as well as smart home devices 104 and/or subscription mobile app 108 for mobile caregivers 106 to inform them of the situation. Smart walker system 10 could even link to services like life alert 110 or home alarm products 112. The intention of smart walker system 10 may be to keep patients, mostly the elderly using their walkers and prevent them from preventable falls which often land them in hospitals or worse.

A feature of the present disclosure may be its ability to alert a forgetful walker user to go back for their walker so they don't fall.

Another feature of the present disclosure may be its ability to assist caregivers by letting them know if a fall risk individual is not using their required walking assistance.

Another feature of the present disclosure may be that the unit would notify the care giver when the battery was running low or the person removed it.

Another feature of the present disclosure may be it could come with an additional unit to go on the person so one would always be charged or a removable battery option.

Another feature of the present disclosure could be a subdermal implant for the user device if it is technically possible.

Another feature of the present disclosure may be its ability to potentially save hospitals and insurers tens of thousands of dollars by preventing walker users from getting hurt.

In the specification and/or figures, typical embodiments of the disclosure have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein but is limited only by the following claims.

1. A smart walker system for a medical assistance walker comprising:
a walker attachment configured to be attached to the medical assistance walker; and
a user proximity sensor in communication with the walker attachment on the medical assistance walker for determining a proximity of the user proximity sensor relative to the walker attachment on the medical assistance walker;
the user proximity sensor is configured to be worn by a user;
the user proximity sensor is configured to detect when the user moves away from the medical assistance walker a set distance;
wherein, when the user wearing the user proximity sensor moves the set distance away from the walker attachment on the medical assistance walker, the smart walker system is configured to alert the user to use the medical assistance walker.

2. The smart walker system of claim 1 configured to promote the use of the medical assistance walker by the user.

3. The smart walker system of claim 2 configured to promote the use of the medical assistance walker to prevent accidental falls.

4. The smart walker system of claim 1, wherein the user proximity sensor is configured to be worn around a neck of the user.

5. The smart walker system of claim 4, wherein the user proximity sensor including:
a wearable device housing a proximity sensor in communication with the walker attachment on the medical assistance walker; and
a flexible elongated member connected at a first side of the wearable device on a first end and connected to a second side of the wearable device on a second end.

6. The smart walker system of claim 5, wherein the wearable device including:
an oval shaped housing; and
a first light in the oval shaped housing configured for indicating a status of the smart walker system.

7. The smart walker system of claim 1, wherein the walker attachment is configured to attach to one of a plurality of support members of the medical assistance walker.

8. The smart walker system of claim 7, wherein the walker attachment is configured to attach to a top front cross member of one of the plurality of support members of the medical assistance walker.

9. The smart walker system of claim 8, wherein the walker attachment is configured to be retrofitted to an existing medical assistance walker, where the walker attachment including an attachment means configured for attaching the walker attachment to the top front cross member, the attachment means comprising:
a hinged back member with hinges configured to wrap around the top front cross member of the medical assistance walker; and
a key lock configured to lock the hinged back member around the top front cross member of the medical assistance walker.

10. The smart walker system of claim 1, wherein the walker attachment including:
a rectangular housing;
a second light in the rectangular housing configured for indicating a device connection status;
a third light in the rectangular housing configured for indicating a device disconnected status;
a proximity distance adjuster configured for adjusting the set distance;
a system display configured to display information of the smart walker system;
or combinations thereof.

11. The smart walker system of claim 10, wherein the walker attachment including:
the rectangular housing;
the second light in the rectangular housing configured for indicating the device connection status in green;
the third light in the rectangular housing configured for indicating the device disconnected status in red;
the proximity distance adjuster configured for adjusting the set distance; and
the system display configured to display information of the smart walker system.

12. The smart walker system of claim 1 further comprising a charger for charging the walker attachment, the user proximity sensor, or combinations thereof.

13. The smart walker system of claim 12, wherein the charger including:
a power cord with a standard plug configured for attaching to an electrical outlet;
a fourth light for indicating charging;
a fifth light for indicating power to the charger;
a first charging port for connecting the walker attachment;
a second charging port for connecting the user proximity sensor;
a sixth light configured for indicating a status of the walker attachment;
a seventh light configured for indicating a status of the user proximity sensor;
or combinations thereof.

14. The smart walker system of claim 13, wherein the charger including:
the power cord with the standard plug configured for attaching to the electrical outlet;
the fourth light for indicating the charging;
the fifth light for indicating the power to the charger;
the first charging port for connecting the walker attachment;
the second charging port for connecting the user proximity sensor;
the sixth light configured for indicating the status of the walker attachment; and
the seventh light configured for indicating the status of the user proximity sensor.

15. The smart walker system of claim 1 configured to remind the user to use the medical assistance walker, to alert nursing stations, to alert smart home devices, to notify a mobile care taker of a subscription mobile app, to notify life alert, to notify a home alarm product, or combinations thereof.

16. The smart walker system of claim 14 configured to remind the user to use the medical assistance walker, to alert nursing statins, to alert smart home devices, to notify a mobile care taker of a subscription mobile app, to notify a life alert, and to notify a home alarm product.

17. The smart walker system of claim 15, wherein the walker attachment including a Wi-Fi communication device configured to communicate over an internet with the nursing stations, to alert the smart home devices, to notify the mobile care taker of the subscription mobile app, to notify the life alert, to notify the home alarm product, or combinations thereof.

18. The smart walker system of claim 16, wherein the subscription mobile app having a user interface including:
an about us tab;
a login tab;
a products tab;
a help tab;
a visual indication of an active walker or an inactive walker;
or combinations thereof.

19. A smart walker system for a medical assistance walker comprising:
a walker attachment configured to be attached to the medical assistance walker, the walker attachment is configured to attach to a top front cross member of the medical assistance walker, the walker attachment is configured to be retrofitted to an existing medical assistance walker, where the walker attachment including an attachment means configured for attaching the walker attachment to the top front cross member, the attachment means comprising:
a hinged back member with hinges configured to wrap around the top front cross member of the medical assistance walker; and
a key lock configured to lock the hinged back member around the top front cross member of the medical assistance walker;
wherein the walker attachment including:
a rectangular housing;
a second light in the rectangular housing configured for indicating a device connection status;
a third light in the rectangular housing configured for indicating a device disconnected status;
a proximity distance adjuster configured for adjusting a set distance; and
a system display configured to display information of the smart walker system;
a user proximity sensor in communication with the walker attachment on the medical assistance walker for determining a proximity of the user proximity sensor relative to the walker attachment on the medical assistance walker;
the user proximity sensor is configured to be worn around a neck of a user;
the user proximity sensor is configured to detect when the user moves away from the medical assistance walker the set distance;
the user proximity sensor including:
a wearable device housing a proximity sensor in communication with the walker attachment on the medical assistance walker, the wearable device including:
an oval shaped housing;
a first light in the oval shaped housing configured for indicating a status of the smart walker system; and
a flexible elongated member connected at a first side of the wearable device on a first end and connected to a second side of the wearable device on a second end; and
a charger for charging the walker attachment, the user proximity sensor, or combinations thereof, the charger including:
a power cord with a standard plug configured for attaching to an electrical outlet;
a fourth light for indicating charging;
a fifth light for indicating power to the charger;
a first charging port for connecting the walker attachment;
a second charging port for connecting the user proximity sensor;
a sixth light configured for indicating the status of the walker attachment; and
a seventh light configured for indicating the status of the user proximity sensor;
wherein, when the user wearing the user proximity sensor moves the set distance away from the walker attachment on the medical assistance walker, the smart walker system is configured to alert the user to use the medical assistance walker;
wherein, the smart walker system is configured to promote the use of the medical assistance walker by the user, and to promote the use of the medical assistance walker to prevent accidental falls; and
wherein, the smart walker system is configured to remind the user to use their walker, to alert nursing stations, to alert smart home devices, to notify a mobile care taker of a subscription mobile app, to notify life alert, to notify a home alarm product, or combinations thereof;
wherein the walker attachment including a Wi-Fi communication device configured to communicate over an internet with the nursing stations, to alert the smart home devices, to notify the mobile care taker of the subscription mobile app, to notify the life alert, to notify the home alarm product, or combinations thereof.

20. A method of alerting a user to use a medical assistance walker comprising:
attaching a walker attachment to the medical assistance walker;
wearing a user proximity sensor by the user;
determining a proximity of the user with the user proximity sensor relative to the medical assistance walker with the walker attachment;
detecting when the user moves away from the medical assistance walker a set distance; and
alerting the user to use the medical assistance walker.
